(19) United States
(12) Patent Application Publication
(10) Pub. No.: US 2023/0100267 A1
(43) Pub. Date: Mar. 30, 2023

(54) INHIBITION OF CELLULAR DYSFUNCTION AND CELL DEATH WITH DEUTERATED POLYUNSATURATED FATTY ACIDS

(71) Applicant: RETROTOPE, INC., Los Altos, CA (US)

(72) Inventors: Peter Milner, Los Alamos, CA (US); Mikhail Sergeevich Shchepinov, Oxford (GB)

(21) Appl. No.: 17/518,507

(22) Filed: Nov. 3, 2021

Related U.S. Application Data
(60) Provisional application No. 63/240,751, filed on Sep. 3, 2021.
(60) Provisional application No. 63/253,690, filed on Oct. 8, 2021.
(60) Provisional application No. 63/253,061, filed on Oct. 6, 2021.

(51) Int. Cl. A61K 31/201; A61P 25/28

(52) U.S. Cl. CPC A61K 31/201; A61P 25/28

(57) ABSTRACT

Disclosed are methods for inhibiting cellular dysfunctionality and subsequent cell death due to cellular accumulation of oxidized polyunsaturated fatty acids (PUFAs) products wherein said accumulation is mediated, at least in part, by impaired enzymatic process(es) that are responsible for neutralizing said oxidized products.

### CROSS-REFERENCE TO RELATED APPLICATIONS

This application claims the benefit of U.S. Provisional Application No. 63/240,751, filed Sep. 3, 2021, U.S. Provisional Application No. 63/253,690, filed Oct. 8, 2021, and U.S. Provisional Application No. 63/253,061, filed Oct. 6, 2021, each of which is incorporated herein by reference in its entirety and for all purposes.

### TECHNICAL FIELD

Disclosed are methods for inhibiting cellular dysfunction and subsequent cell death due to cellular accumulation of oxidized polyunsaturated fatty acids (PUFAs) products. In one embodiment, the accumulation of oxidized PUFA products is mediated, at least in part, by impaired enzymatic process(es) that are responsible for neutralizing said oxidized products. In one embodiment, the disclosed methods comprise incorporating deuterated arachidonic acid into said cells and components thereof in sufficient amounts to reduce the generation of said oxidized products to a level wherein said impaired enzymatic process(es) are capable of neutralizing a sufficient level of the reduced amounts of oxidized products so generated.

In one embodiment, the subsequent cell death occurs via a regulated cell death pathway.

### BACKGROUND

Cellular dysfunctionality occurs when one or more cellular perturbations disrupt the ability of the cell to retain stasis. When such perturbations continue unabated, the cellular damage caused by the perturbations becomes non-recoverable, leading to cell death. One major cause of cellular dysfunction, particularly in neurodegenerative diseases, is the generation of oxidized PUFA products in amounts that exceed the ability of regulatory cellular enzymes to neutralize such products. In some cases, this inability is associated with impairments to these enzymes that can be aged related or genetically related. In other cases, this inability is associated with increased amounts of oxidized PUFA products generated in the patient, resulting in an imbalance where the increased amounts of these products exceed the neutralizing capacity of the regulatory enzymes.

Multiple different cell membranes comprise phospholipids containing at least one PUFA. Moreover, these phospholipids are stacked together and each is in intimate contact with its adjacent member. The structure of all PUFAs include a cis-1,4-diene system with varying numbers of unsaturated sites, a bis-allylic methylene group separating the double bonds found at a 1,4-position, and mono-allylic positions flanking both ends of the polyunsaturated chain. Such a structure is represented below showing both the bis-allylic methylene group and the mono-allylic methylene groups, as well as the characteristic unsaturation at positions 1 and 4.

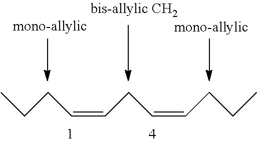

The hydrogen atoms of these bis-allylic methylene groups are particularly susceptible to oxidizing agents. When oxidized, a cascade of autooxidation occurs where a first oxidized PUFA can initiate oxidation of a neighboring PUFA which, in turn, can initiate oxidation of its neighboring PUFA creating a process called Lipid Peroxidation (LPO). These oxidized PUFA are transformed into a number of toxic metabolites, examples of which are described below. Both the oxidized PUFAs and their toxic metabolites are referred to herein as “oxidized PUFA products”.

The toxicity of oxidized PUFA products generated by LPO is well established. Oxidation occurs in the phospholipid portion of a biological membrane. Once oxidized, enzymes such as A2 phospholipase remove the oxidized PUFA, followed by neutralization of the oxidized product by a number of endogenous enzymatic antioxidants including superoxide dismutase (SOD), catalase (CAT) and glutathione peroxidase (GPx), and thioredoxin (Trx). Impairment of any of these enzymes can lead to an accumulation of non-neutralized oxidized products which, in turn, causes the toxicity. For example, infantile neuroaxonal dystrophy (INAD) is caused by genetic defects in the A2 phospholipase enzyme, limiting its ability to remove (hydrolyze) oxidized arachidonic acids at the SN2 position of the phospholipid.

As these oxidative perturbations continue, cellular dysfunction often occurs prior to cell death. Continuation of these perturbations leads to cell death via a regulated cell death (RCD) pathway. These RCD pathways are well recognized in the art and include, by way of example only, intrinsic apoptosis, extrinsic apoptosis, mitochondrial permeability transition (MPT)-driven necrosis, necroptosis, oxytosis, ferroptosis, and pyroptosis to name a few. In each case, cell death is the result of unrecoverable cellular perturbations that terminate cell survival. See, e.g., Galluzzi, et al., Nature, 25:486-541 (2018).

Oxidizing perturbations associated with the accumulation of oxidized PUFA products have been implicated in a wide variety of diseases including mitochondrial diseases, neurodegenerative diseases (including neurodegenerative muscle diseases), retinal diseases, energy processing disorders, cardiac diseases, to name a few.

Enzymatic impairment can be due to genetic defects which limit enzymatic activity below that necessary to neutralize oxidized PUFA products. Alternatively, enzymatic impairment can be age related. In one example, the amounts of enzymes expressed and/or the activity of these expressed enzymes are reduced as one ages. Still further, enzymatic impairment can also be due to the inability of the cell to produce sufficient enzyme to counter an increasing amount of oxidized arachidonic acid products arising from a diseased condition. Regardless of the cause of such impairment, the impaired enzymes are capable of neutralizing only a portion of the oxidized products, thereby allowing for the intracellular accumulation of these products. As the amount of these oxidized products accumulate, the functionality of the cell is first compromised. For example, if the cell is a neuron and the disease is INAD, the loss of functionality due to impairment of the A2 phospholipase enzyme will eventually impact the patient's ability to swallow as well as other vital functions.

Loss of cellular function is followed by cellular death due to any one of the RCD pathways when the cellular perturbation remains unabated.

In view of the above, methods that addressed these diseases by addressing the underlying impairment of enzymatic conditions are urgently needed. Preferably, such methods would substantially stop disease progression and then reverse at least a portion of the lost cellular functionality.

### SUMMARY

Generally, disclosed are methods for inhibiting cellular dysfunction and subsequent cell death. In many cases, cellular dysfunction and subsequent cellular death is associated with the cellular accumulation of oxidized PUFA products. Without being limited to any theory, such accumulation is believed to arise as a result of impaired enzymatic process(es) that prevent the neutralization of all of such oxidized products. Still further, reducing the amount of oxidized PUFA products generated in the cell would allow for the neutralization of a much larger percentage of the oxidized products so generated.

The methods described herein comprise incorporating deuterated arachidonic acid into cells in sufficient amounts to limit the amount of oxidized arachidonic acid generated to a level that said impaired enzymatic processes are capable of neutralizing a sufficient amount (e.g., substantially all) of said oxidized products to treat the disease. In vivo, the deuterated arachidonic acid is preferably generated from a prodrug such as a deuterated linoleic acid or ester thereof or an ester of a deuterated arachidonic acid. In one embodiment, the prodrug is 11,11-D2-linoleic acid or an ester thereof. In vivo, 11,11-D2-linoleic acid or an ester thereof is converted to 13,13-D2-arachidonic acid which is incorporated into cells. Both the reduction in the amount of oxidized PUFA products generated and the ability of regulatory enzymes to neutralize the reduced amounts of oxidized products still generated allow for inhibiting further dysfunction and cell death in the treated cells.

In one embodiment, there is provided a method for treating a patient with a neurodegenerative disease which method comprises administering to said patient sufficient amounts of deuterated linoleic acid (e.g. 11,11-D2-linoleic acid) or an ester thereof as a prodrug for deuterated arachidonic acid (e.g. 13,13-D2-arachidonic acid) for a sufficient period of time to reach a concentration of deuterated arachidonic acid in the red blood cells of at least about 12% (based on the total amount of arachidonic acid present), thereby treating said disease.

In one embodiment, there is provided a method for inhibiting cellular dysfunction and subsequent cell death due directly or indirectly to cellular accumulation of oxidized PUFA products as a result of impaired enzymatic process(es) that limit the neutralization of said oxidized products, said method comprises incorporating deuterated arachidonic acid into said cell and components thereof in sufficient amounts to reduce the amount of oxidized PUFAs generated to a level that said impaired enzymatic processes are capable of neutralizing substantially all of said oxidized products produced, thereby inhibiting cellular dysfunction and subsequent cell death.

In one embodiment, the impairment responsible for reducing the ability of regulatory enzymes responsible for neutralizing oxidized PUFA products may be due to genetic defects leading to impaired enzymes with limited activity. The impairment may also be due to age related limitations on the amount of enzyme expressed and/or a reduction in the activity of the enzymes so expressed. The impairment may also be due to the inability of the cell to produce sufficient enzyme to counter an increasing amount of oxidized PUFA products arising from a disease condition. Finally, the impairment may be due to a combination of two or more of these factors.

In one embodiment, there is provided a method for stabilizing at least one vital function in a patient with a neurodegenerative disease wherein the at least one vital function has been degraded as a result of said disease, said method comprises:
administering to said patient a sufficient amount of deuterated linoleic acid or an ester thereof as a prodrug for deuterated arachidonic acid for a sufficient period of time such that said deuterated arachidonic acid is incorporated into a neuron; and
continuing said administration of said deuterated linoleic acid or ester thereof until the concentration of deuterated arachidonic acid in red blood cells from the patient is at least about 12 percent based on the total amount of arachidonic acid in the red blood cells, whereupon at least one vital function in said patient has been stabilized.

In one embodiment, there is provided a method for extending the life expectancy of a patient with a neurodegenerative disease, said method comprises:
administering to said patient a sufficient amount of deuterated linoleic acid or an ester thereof as a prodrug for deuterated arachidonic acid for a sufficient period of time such that said deuterated arachidonic acid is incorporated into the at-risk neurons of the patient as well as in the red blood cells; and
continuing said administration of said deuterated linoleic acid or ester thereof until the concentration of deuterated arachidonic acid in red blood cells from the patient is at least about 12 percent based on the total amount of arachidonic acid in the red blood cells;
maintaining said concentration of said deuterated arachidonic acid such that the life expectancy of said patient is extended.

In one embodiment, the deuterated linoleic acid or ester thereof is 11,11-D2-linoleic acid or an ester thereof. A fraction of this compound is converted in vivo to 13,13-D2-arachidonic acid and that fraction of 11,11-D2-linoleic acid acts as a prodrug of 13,13-D2-arachidonic acid.

In one embodiment, the deuterated linoleic acid or ester thereof is 8,8,11,11-D4-linoleic acid or an ester thereof. A fraction of this compound is converted in vivo to 10,10,13,13-D4-arachidonic acid and that fraction of 8,8,11,11-D2-linoleic acid acts as a prodrug of 10,10,13,13-D4-arachidonic acid.

In one embodiment, said administration to said patient of said deuterated linoleic acid or an ester thereof as a prodrug for a deuterated arachidonic acid is further continued until at least one vital function in said patient is improved.

In one embodiment, the neurodegenerative disease treated in the patient is mediated, directly or indirectly, by neural accumulation of oxidized PUFA products as a result of impaired enzymatic process(es) that limit the amount of said oxidized products that can be neutralized. In one embodiment, said method comprises administering to a patient a sufficient amount of deuterated linoleic acid (e.g. 11,11-D2-linoleic acid) or an ester thereof as a prodrug for deuterated arachidonic acid (e.g. 13,13-D2-arachidonic acid) for a sufficient period of time such that the concentration of 13,13-D2-arachidonic acid in red blood cells ranges from about 12% to about 25% based on the total amount of arachidonic acid (including deuterated arachidonic acid), and preferably from about 15% to about 20%. When such a concentration is reached, the amount of oxidized PUFA products generated is reduced to a level that said impaired enzymatic processes are capable of neutralizing a greater percentage of said oxidized products, thereby treating said disease.

In one embodiment, loss of cellular function is followed by cell death which, in turn, is the result of a regulated cell death pathway. In another embodiment, the regulated cell death pathway is selected from the group consisting of intrinsic apoptosis, extrinsic apoptosis, mitochondrial permeability transition (MPT)-driven necrosis, necroptosis, oxytosis, ferroptosis, and pyroptosis.

### BRIEF DESCRIPTION OF THE DRAWINGS

FIG. 1 illustrates a concentration graph for the amount of 13,13-D2-deuterated arachidonic acid in the red blood cells (RBCs) in a patient at different points in time from the start of therapy and then extrapolated back to 1 month after start of therapy.

FIG. 2 is a Kaplan Meier Curve comparing the survival of treated patients versus untreated patients over a prolonged period of time.

FIGS. 3A and 3B are graphs of deuterated fatty acid levels in the red blood cells and plasma during administration of 11,11-D2-linoleic acid ester. FIG. 3A illustrates the concentrations of 11,11-D2-linoleic acid in plasma and red blood cells over time. FIG. 3B illustrates the concentrations of 13,13-D2-arachidonic acid in plasma and red blood cells over time.

### DETAILED DESCRIPTION

Disclosed are methods for stabilizing at least one vital function in a patient with a neurodegenerative disease due to cellular accumulation of oxidized PUFA products as a result of impaired enzymatic process(es) that limit the neutralization of said oxidized products.

Prior to discussing this invention in more detail, the following terms will first be defined. Terms that are not defined are given their definition in context or are given their medically acceptable definition.

The terminology used herein is for the purpose of describing particular embodiments only and is not intended to be limiting of the invention. As used herein, the singular forms “a”, “an” and “the” are intended to include the plural forms as well, unless the context clearly indicates otherwise.

As used herein, the term “optional” or “optionally” means that the subsequently described event or circumstance can or cannot occur, and that the description includes instances where the event or circumstance occurs and instances where it does not.

As used herein, the term “about” when used before a numerical designation, e.g., temperature, time, amount, concentration, and such other, including a range, indicates approximations which may vary by (+) or (−) 15%, 10%, 5%, 1%, or any subrange or subvalue there between. Preferably, the term “about” when used with regard to a dose amount means that the dose may vary by +/−10%.

As used herein, the term “comprising” or “comprises” is intended to mean that the compositions and methods include the recited elements, but not excluding others.

As used herein, the term “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention.

As used herein, the term “consisting of” shall mean excluding more than trace elements of other ingredients and substantial method steps. Embodiments defined by each of these transition terms are within the scope of this invention.

As used herein, linoleic acid has the numbering system as described below:

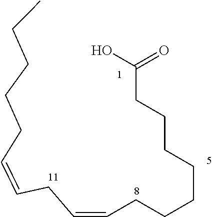 [SMILES: CCCCC/C=C\C/C=C\CCCCCCCC(=O)O]

As used herein, the term “deuterated linoleic acid or an ester thereof” refers to any deuterated linoleic acid or ester thereof that comprises at least one deuterium atom at a carbon atom that, upon conversion to arachidonic acid, is found at a bis-allylic site of arachidonic acid. Preferred deuterated linoleic acids or esters thereof include 11,11-D2-linoleic acid or an ester thereof, 8,8-D2-linoleic acid or an ester thereof, or 8,8,11,11-D4-linoleic acid or an ester thereof. Upon in vivo conversion, the 8,8-D2-linoleic acid is transformed into 10,10-D2-arachidonic acid; 11,11-D2-linoleic acid is transformed into 13,13-D2-arachidonic acid; whereas the 8,8,11,11-D4-linoleic acid becomes 10,10,13,13-D4-arachidonic acid. Where 11,11-D2-linoleic acid is referred to herein, it is to be understood that any deuterated linoleic acid or an ester thereof having two deuterium atoms at the 11 position thereof is included regardless of the presence of deuterium atoms at other sites in the molecule, unless otherwise stated.

As used herein, arachidonic acid has the numbering system as described below:

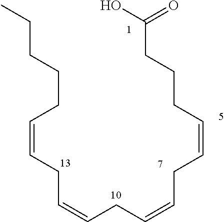 [SMILES: CCCCC/C=C\C/C=C\C/C=C\C/C=C\CCCC(=O)O]

where each of positions 7, 10 and 13 are bis-allylic positions within the structure. It is noted that the 10 position of arachidonic acid was previously the 8 position of linoleic acid or clearly implied.

As used herein and unless the context dictates otherwise, the term “deuterated arachidonic acid or an ester thereof” refers to any arachidonic acid that comprises at least one deuterium atom at a bis-allylic site of carbon atom. Preferred deuterated arachidonic acid or esters thereof include 7-D1-arachidonic acid or an ester thereof; 10-D1-arachidonic acid or an ester thereof; 13-D1-arachidonic acid or an ester thereof; 7,10-D2-arachidonic acid or an ester thereof; 7,13-D2-arachidonic acid or an ester thereof; 10,13-D2-arachidonic acid or an ester thereof; 7,7-D2-arachidonic acid or an ester thereof; 10,10-D2-arachidonic acid or an ester thereof; 13,13-D2-arachidonic acid or an ester thereof; 7,10,13-D3-arachidonic acid or an ester thereof; 7,7,10-D3-arachidonic acid or an ester thereof; 7,10,10-D3-arachidonic acid or an ester thereof; 7,13,13-D3-arachidonic acid or an ester thereof; 10,10,13-D3-arachidonic acid or an ester thereof; 10,13,13-D3-arachidonic acid or an ester thereof; 7,7,10,13-D4-arachidonic acid or an ester thereof; 7,7,10,10-D4-arachidonic acid or an ester thereof; 7,10,10,13-D4-arachidonic acid or an ester thereof; 7,10,13,13-D4-arachidonic acid or an ester thereof; 7,7,13,13-D4-arachidonic acid or an ester thereof; 10,10,13,13-D4-arachidonic acid or an ester thereof; 7,7,10,10,13-D5-arachidonic acid or an ester thereof 7,7,10,13,13-D5-arachidonic acid or an ester thereof; 7,10,10,13,13-D5-arachidonic acid or ester thereof; 7,7,10,10,13,13-D6-arachidonic acid or ester thereof or mixtures of any two or more. Where 13,13-D2-arachidonic acid is referred to herein, it is to be understood that any deuterated arachidonic acid or an ester thereof having two deuterium atoms at the 13 position thereof is included regardless of the presence of deuterium atoms at other sites in the molecule, unless otherwise stated or clearly implied.

Preferred D2-arachidonic acids include 7,7-D2-arachidonic acid or esters thereof; 10,10-D2-arachidonic acid or esters thereof; and 13,13-D2-arachidonic acid or esters thereof.

Preferred D4-arachidonic acids or esters thereof include 7,7,10,10-D4-arachidonic acid or esters thereof; 7,7,13,13-D4-arachidonic acid or esters thereof; and 10,10,13,13-D4-arachidonic acid or esters thereof. In one embodiment, 10,10,13,13-D4-arachidonic acid can be biosynthesized from 8,8,11,11-D4-gamma linolenic acid or from 10,10,13,13-D6-d-homa-gamma linolenic acid. The bioconversion of both of these PUFAs results in 10,10,13,13-D4-arachidonic acid. Both the 8,8,11,11-D4-gamma linolenic acid or the 10,10,13,13-D6-d-homa-gamma linolenic acid (or esters of either) can be prepared by ruthenium catalysis as described below provided that such will result in at least 80% deuteration of their bis-allylic positions as well as nominal amounts of deuteration at one or both of the mono-allylic positions (e.g., less than about 25%).

Preferred D6-arachidonic acid includes 7,7,10,10,13,13-D6-arachidonic acid or esters thereof including compositions of deuterated arachidonic acid or ester thereof that comprises, on average, at least about 80% of the hydrogen atoms at each of the bis-allylic sites having been replaced by deuterium atoms and, on average, no more than about 35% of the hydrogen atoms at the mono-allylic sites having been replaced by deuterium atoms. For example, in the case of 80% deuteration of the 3 bis-allylic sites and 35% deuteration of the mono-allylic sites, the total amount of deuterium is (6×0.8)+(4×0.35)=6.2 exclusive of the naturally occurring amount of deuterium in each of the remaining methylene and methyl groups within the structure.

In some embodiments, the amount of deuterium replacing hydrogen at the bis-allylic sites (7, 10 and 13) and at the mono-allylic sites (4 and 16) of arachidonic acid can be any one of the following:
at least about 85% deuterium at bis-allylic sites/no more than about 30% at mono-allylic sites;
at least about 85% deuterium at bis-allylic sites/no more than about 25% at mono-allylic sites;
at least about 85% deuterium at bis-allylic sites/no more than about 20% at mono-allylic sites;
at least about 85% deuterium at bis-allylic sites/no more than about 10% at mono-allylic sites;
at least about 85% deuterium at bis-allylic sites/no more than about 5% at mono-allylic sites;
at least about 90% deuterium at bis-allylic sites/no more than about 30% at mono-allylic sites;
at least about 90% deuterium at bis-allylic sites/no more than about 25% at mono-allylic sites;
at least about 90% deuterium at bis-allylic sites/no more than about 20% at mono-allylic sites;
at least about 90% deuterium at bis-allylic sites/no more than about 10% at mono-allylic sites;
and
at least about 90% deuterium at bis-allylic sites/no more than about 5% at mono-allylic sites.

As used herein and unless the context dictates otherwise, the term “an ester thereof” refers to a C₁-C₆ alkyl esters, glycerol esters (including monoglycerides, diglycerides and triglycerides), sucrose esters, phosphate esters, and the like. The particular ester group employed is not critical provided that the ester is pharmaceutically acceptable (non-toxic and biocompatible). In one embodiment, the ester is a C₁-C₆ alkyl ester which is preferably an ethyl ester.

As used herein, the term a “prodrug of deuterated arachidonic acid” refers to deuterated linoleic acid or an ester thereof (e.g., 11,11-D2-linoleic acid or an ester thereof) or to esters of deuterated arachidonic acid (e.g., esters of 13,13-D2-arachidonic acid). Esters of either deuterated linoleic acid or deuterated arachidonic acid are rapidly hydrolyzed in the gastrointestinal tract to provide for the free acid. In the case of deuterated linoleic acid, at least a portion of this compound is converted in vivo to deuterated arachidonic acid.

As used herein, the term “phospholipid” refers to any and all phospholipids that are components of the cell membrane or other lipid membrane(s) within a cell. Included within this term are phosphatidylcholine, phosphatidylethanolamine, phosphatidylserine, and sphingomyelin. In the motor neurons, the cell membrane is enriched in phospholipids comprising arachidonic acid.

The term “bis-allylic site” refers to the methylene group (CH₂) separating two double bonds as depicted above.

The term “mono-allylic site” refers to the methylene group having an adjacent neighboring double bond on one side and a further methylene group on the opposite side as depicted above.

The term “cellular component” refers to any relevant structure found in human cells, including organelles having a lipid or phospholipid wall. Examples of such cellular components include, by way of example only, the mitochondria, the endoplasmic reticulum, golgi apparatus, nuclear membrane, and the like. Cellular components also include the cell membrane (plasma membrane).

The term “regulatory enzymes” as it relates to the neutralization of oxidized arachidonic acid products refer to those enzymes that are responsible to remove, alter, or destroy one or more of the oxidized arachidonic acid products from the cells to prevent the accumulation of these oxidized products within the cell. Examples of regulatory enzymes include glutathione peroxidase (GPx) enzymes, particularly GP×4. When these enzymes are impaired, such impairment results in the accumulation of lipid peroxides leading to loss of functionality, which is often death followed by cell death. Gaschler, et al., Biochem. Biophys. Res. Commun., 482(3):419-425 (2017).

The term “oxidized PUFA products” refer to any oxidized form of a polyunsaturated fatty acid as well as any and all metabolites formed from the oxidized PUFA including reactive aldehydes, ketones, alcohols, carboxyl derivatives which are toxic to the cell where found in a phospholipid, a lipid bilayer, or as an enzyme substrate.

The term “cellular dysfunctionality” or “cellular dysfunction” refers to a cell's inability to properly function in conjunction with a population of similar cells that control essential/vital functions of the body such as memory, motor skills, breathing, swallowing, and the like. As more and more cells within this population exhibit cellular dysfunctionality, the overall dysfunctionality manifests itself in diminished capacity for such functions. Loss of motor skills are symptoms of ALS, ataxia, Huntington's Disease, INAD, and neuropathy, to name a few. Loss of memory function is symptomatic of Alzheimer's disease (AD) and other forms of dementia.

The term “impairment” as it relates to an enzyme and an enzymatic process refers to the inability of one or more enzymes responsible for neutralizing oxidized arachidonic acid products to adequately prevent accumulation of said oxidized products. For example, the impairment may be due to genetic defects leading to impaired enzymes with limited activity. The impairment may also be due to age related limitations on the amount of enzyme expressed and/or a reduction in the activity of the enzymes so expressed. The impairment may also be due to the inability of the cell to produce sufficient enzyme to counter an increasing amount of oxidized arachidonic acid products arising from a diseased condition. Finally, the impairment may be due to a combination of one or more of these factors.

The term “neutralization of oxidized products” refers to enzymatic processes that remove, alter, or destroy toxic oxidized PUFA products from the cells to prevent the accumulation of these oxidized products within the cell. In healthy individuals, the neutralization of the oxidized PUFA products prevents accumulation of these products thereby assuring that dysfunctionality of cells especially neurodegenerative cells is controlled.

The term “stabilizing at least one vital function” refers to a reduction in the rate of loss of at least one vital function such as breathing, swallowing, neck control, and the like to an annualized rate of loss of less than 20% per year for that vital function, as compared to the rate of loss determined for the natural history of that the patient or the average rate of loss for a cohort of patients.

The term “improving at least one vital function” refers to any improvement observed in at least one vital function of a patient during the course of treatment. In one embodiment, the improvement can be measured by the ability to regain partial or full neck control during the course of treatment per the methods described herein. In another embodiment, the improvement in swallowing can be measured by the number of aspirations over a given time period as compared to the number of aspirations found during the natural history. Such an improvement can be measured in a given patient or in a cohort of patients. As per the examples below, a child with INAD having substantially no neck control at the start of therapy, evidences incremental neck control at 4 months after start of therapy followed by complete neck control (although temporary) by 10 months after start of therapy. Typically, any improvement in a vital functionality will be first observed about 4, or 6 or 12 months after the start of therapy. In the case of aspiration, a reduction in the annualized number of aspirations is reduced by at least 20%, preferably at least 25% and more preferably at least 50%.

The term “improving the life expectancy” refers to a statistically significant change in the life of patients treated as per the methods of this invention as compared to control who are not so treated. One measure of such improvement is provided by approximately how much longer treated patients leave per year of treatment as compared to control.

As used herein, the term “pathology of a disease” refers to the cause, development, structural/functional changes, and natural history associated with that disease. Included in the pathology of the disease is the reduction in cellular functionality. The term “natural history” means the progression of the disease in the absence of treatment per the methods described herein, for example, prior to the commencement of treatment as described herein.

As used herein, the term “reduced rate of disease progression” means that the rate of disease progression is attenuated after initiation of treatment as compared to the patient's natural history. In one case, the rate of reduction in disease progression using the methods described herein results in a percentage reduction of at least 75% lower or at least 90% lower at a time point after initiation of therapy when compared to the natural history of the patient. The time point may be any time after initiation of therapy, such as at 3 or 4 or 6 or 12 months. Such reduced rates of disease progression evidence that the level of enzymatic control of the oxidized PUFA products.

“Disease progression” as used herein refers to the increase in symptoms associated with the disease over time. Measurement of disease progression for a given disease is within the skill of a clinician. For example, disease progression may be measured by a set of standard questions. In ALS, these standard questions are set forth in Revised ALS Functional Rating Scale. This scale provides a list of 12 questions regarding functionality of the patient. Each question has a value of 0 to 4 with 0 being non-functional and 4 being most functional. The attending clinician evaluates the patient pre-therapy to assess the current status of the patient and the rate of loss of functionality from the onset of the disease and annualizes it (natural history rate of loss or “A”). The patient is then reevaluated at a set period of time after initiation of the therapy to evaluate functionality and the annualized rate of loss is determined (therapy rate of loss or “B”). “A” is then compared to “B”. For example, the reduction in the rate of loss “C” is provided as follows;

C=[(A−B)/A]×100

So if “A” has a value of 60% for the natural history rate of loss and B has a value of 40%, then C is 33% (60-40)/60×100.

The term “therapeutic concentration” means an in vivo concentration of a deuterated arachidonic acid that stabilizes the rate of disease progression and/or progression of at least one vital function of the treated patient as defined herein. Since measuring the concentration of a deuterated arachidonic acid in the motor neurons or in the spinal fluid of a patient is either not feasible or optimal, the therapeutic concentration may be based on the concentration of deuterated arachidonic acid found in red blood cells as provided in the Examples below, as well as in U.S. Provisional Patent Application Ser. No. 63/177,794, which is incorporated herein by reference in its entirety. Accordingly, any reference made herein to a therapeutic concentration of deuterated arachidonic acid is made by evaluating its concentration in red blood cells.

In one embodiment, a concentration of deuterated arachidonic acid in red blood cells of from about 12% to about 30% based on the total amount of arachidonic acid found therein (including deuterated arachidonic acid) has been shown to be therapeutic at the levels set forth herein. In one preferred embodiment, the therapeutic concentration of deuterated arachidonic acid will range from about 15% to about 20% in red blood cells.

As used herein, the term “patient” refers to a human patient or a cohort of human patients suffering from a neurodegenerative disease treatable by administration of a prodrug of deuterated arachidonic acid. The term “adult patient” refers to a subject over 18 years of age and preferably weighing at least about 40 kg at the start of therapy, who is suffering from a neurodegenerative disease treatable by administration of a prodrug of deuterated arachidonic acid. The term “juvenile patient” refers to a patient who is not an adult and preferably weighs no more than about 35 kg at the start of therapy.

It is well known that only a fraction of the linoleic acid consumed per day is converted by the patient into arachidonic acid. The specific amount of arachidonic acid generated depends on the patient's physiology as well as the amount of PUFAs consumed per day. The higher the PUFA load consumed by the patient translates into a smaller amount of deuterated arachidonic acid generated in vivo. Accordingly, in some embodiments the method may comprise limiting the consumption of PUFAs by the patient.

In one embodiment, the term “loading amount” or “primer amount” of deuterated linoleic acid or ester thereof refers to a sufficient amount of deuterated linoleic acid or an ester thereof (e.g., 11,11-D2-linoleic acid or an ester thereof) that provides for in vivo conversion into therapeutic concentration of deuterated arachidonic acid (e.g., 13,13-D2-arachidonic acid). In one embodiment, the loading dose for an adult patient is between about 6 grams and about 12 grams, for example about 9 grams, per day of 11,11-D2-linoleic acid ethyl ester (e.g., about 8.55 grams when accounting for the ethyl ester which will be removed and a small percent of impurities) for at least 30 days. In a child, the loading dose is between about 2.5 grams and about 4.5 grams per day, for example about 3.9 grams per day or about 3 grams per day. Afterwards, the duration of the loading dose is optionally continued or the dosing can be increased by the attending clinician depending on whether the analysis of deuterated arachidonic acid in the red blood cells evidences insufficient levels of deuterated arachidonic acid at about 30 days post start of therapy as compared to, e.g., a standard curve.

The methods described herein are based, in part, on the discovery that the primer doses of deuterated linoleic acid or an ester thereof employed to date are well tolerated by patients and provide for rapid onset of a sufficient in vivo concentration of deuterated arachidonic acid to provide for a reduced and stabilized rate of disease progression.

As used herein, the term “maintenance dose” refers to a dose of deuterated linoleic acid or an ester thereof (e.g., 11,11-D2-linoleic acid or an ester thereof) that is less than the primer dose and is sufficient to maintain an in vivo therapeutic concentration of deuterated arachidonic acid in the cell membrane of neurons (e.g., motor neurons), for example, as measured by the amount in the cell membrane of red blood cells. In one embodiment, the maintenance dose of 11,11-D2-linoleic acid or ester thereof for an adult is between about 3 grams and about 8 grams, for example about 5 grams, per day (e.g., about 4.75 grams when accounting for the ethyl ester which will be removed and a small percent of impurities). In one embodiment, the maintenance dose for a child (less than 60 kg) ranges from about 1 to 4 grams and preferably about 2 or about 2.9 grams per day of 11,11-D2-linoleic acid or ester thereof. The maintenance dose may be initiated about 30 days after start of the primer dose.

As used herein, the term “periodic dosing” refers to a dosing schedule that substantially comports to the dosing described herein. Stated differently, periodic dosing includes a patient who is compliant at least 75 percent of the time over a 30-day period and preferably at least 80% compliant with the dosing regimen described herein. In embodiments, the dosing schedule contains a designed pause in dosing. For example, a dosing schedule that provides dosing 6 days a week is one form of periodic dosing. Another example is allowing the patient to pause administration for from about 3 or 7 or more days (e.g., due to personal reasons) provided that the patient is otherwise at least 75 percent compliant. Also, for patients who transition from the loading dose to the maintenance dose, compliance is ascertained by both the loading dose and the maintenance dose.

The term “cohort” refers to a group of at least 2 patients whose results are to be averaged.

As used herein, the term “pharmaceutically acceptable salts” of compounds disclosed herein are within the scope of the methods described herein and include acid or base addition salts which retain the desired pharmacological activity and is not biologically undesirable (e.g., the salt is not unduly toxic, allergenic, or irritating, and is bioavailable). When the compound has a basic group, such as, for example, an amino group, pharmaceutically acceptable salts can be formed with inorganic acids (such as hydrochloric acid, hydroboric acid, nitric acid, sulfuric acid, and phosphoric acid), organic acids (e.g., alginate, formic acid, acetic acid, benzoic acid, gluconic acid, fumaric acid, oxalic acid, tartaric acid, lactic acid, maleic acid, citric acid, succinic acid, malic acid, methanesulfonic acid, benzenesulfonic acid, naphthalene sulfonic acid, and p-toluenesulfonic acid) or acidic amino acids (such as aspartic acid and glutamic acid). When the compound has an acidic group, such as for example, a carboxylic acid group, it can form salts with metals, such as alkali and earth alkali metals (e.g., Na⁺, Li⁺, K⁺, Ca²⁺, Mg²⁺, Zn²⁺), ammonia or organic amines (e.g., dicyclohexylamine, trimethylamine, trimethylamine, pyridine, picoline, ethanolamine, diethanolamine, triethanolamine) or basic amino acids (e.g., arginine, lysine, and ornithine). Such salts can be prepared in situ during isolation and purification of the compounds or by separately reacting the purified compound in its free base or free acid form with a suitable acid or base, respectively, and isolating the salt thus formed.

### Pathology

The resulting pathology of each of the neurodegenerative diseases is different from the underlying etiology of the disease. That is to say that whatever divergent conditions trigger each of these neurodegenerative diseases (the etiology), once triggered the pathology of these diseases involves the accumulation of oxidized PUFA products in affected neurons (“at-risk neurons”). As the amounts of oxidized products increases, the affected neurons lose functionality (become dysfunctional), further extending the pathology of the disease. Ultimately, the damage to the cell arising from increasing concentrations of oxidized PUFA products reaches a point where the cell is non-recoverable and the cell initiates a cell death pathway (CDP). There are a number of recognized CDPs including intrinsic apoptosis, extrinsic apoptosis, mitochondrial permeability transition (MPT)-driven necrosis, necroptosis, oxytosis, ferroptosis, and pyroptosis as well as initiation of the 15-HpETE-PE death signal in the case of 13-oxidized arachidonic acid products. See, Sun et al., Nature Chemical Biology, 2021 which is incorporated by reference in its entirety.

As per the above, the increasing concentration of oxidized PUFA products in the neurons evidence an inability of the regulatory enzymes to neutralize these products. As the unabated accumulation of the oxidized PUFA products increases within at-risk neurons, so too does disease progression. The methods described herein that entail in vivo delivery of deuterated arachidonic acid thereby limiting the amount of oxidized PUFA products generated, have a positive impact on treating the disease.

Neurodegenerative diseases that respond to treatment with deuterated arachidonic acid, whether generated in vivo from deuterated linoleic acid (e.g., 11,11-D2-linoleic acid) or by delivery of deuterated arachidonic acid (e.g., 7,7,10,10,13,13-D6 arachidonic acid), include amyotrophic lateral sclerosis (ALS), tauopathy (including progressive supernuclear palsy—PSP), Friedrich's ataxia, Huntington's Disease, Corticobasal disorder (CBD), Frontotemporal dementia (FTD), Nonfluent variant primary progressive aphasia (nfvPPA), INAD, APO-e4 Alzheimer's Disease, and late onset Tay-Sachs.

The origin of the oxidative stress responsible for peroxidation varies due to differences in the underlying etiology but typically involve one or more different reactive oxygen species. Regardless of the differences in etiology, the production of oxidized PUFA products evidence an imbalance between routine production and detoxification (neutralization) of these oxidized products. The lipid membrane of the cell as well as that of the endoplasmic reticulum and mitochondria of neurons are highly enriched in arachidonic acid (a 20-carbon chain polyunsaturated fatty acid (“PUFA”) having 4 sites of cis-unsaturation). Separating each of these 4 sites are 3 bis-allylic methylene groups. These groups are particularly susceptible to oxidative damage as compared to allylic methylene and methylene groups. Apart from leading to further membrane damage, oxidation of arachidonic acid reduces the local concentration of arachidonic acid and must be replaced. Thus, it is a double hit: a positive bioactive membrane component is converted to a toxic membrane component.

Given that the neurons have a high concentration of arachidonic acid in their lipid membranes, replacement of damage or lost arachidonic acid in these membranes with deuterated arachidonic acid reinforces these structures in the cell and protects against formation of oxidized lipid products. For example, once a bis-allylic methylene group in one arachidonic acid is oxidized by a ROS, a cascade of further oxidation of other arachidonic acid groups in the lipid membrane occurs. This is because a single ROS generates oxidation of a first arachidonic acid component through a free radical mechanism which, in turn, can oxidize a neighboring arachidonic acid through the same free radical mechanism which yet again can oxidize another neighboring arachidonic acid in a process referred to as lipid chain auto-oxidation. The resulting damage includes a significant number of oxidized arachidonic acid products in the cell membrane and in the membrane of organelles. However, if an oxidized arachidonic acid is adjacent to the deuterated arachidonic acid, then that deuterated arachidonic acid acts as a chain-reaction terminator due to the much higher stability of the carbon-deuterium bond against oxidation as compared to the carbon-hydrogen bond.

Oxidized arachidonic acid and other oxidized PUFA products negatively affect the fluidity and permeability of cell membranes in the patient's neurons. In addition, they can lead to oxidation of membrane proteins as well as be converted into a large number of highly reactive carbonyl compounds. The latter include reactive species such as acrolein, malonic dialdehyde, glyoxal, methylglyoxal, etc. (Negre-Salvayre A, et al. Brit. J. Pharmacol. 2008; 153:6-20). The most prominent products of arachidonic acid oxidation are alpha, beta-unsaturated aldehydes such as 4-hydroxynon-2-enal (4-HNE; formed from n-6 PUFAs like LA or AA), and corresponding ketoaldehydes (Esterfbauer H, et al. Free Rad. Biol. Med. 1991; 11:81-128). As noted above, these reactive carbonyls cross-link (bio)molecules through Michael addition or Schiff base formation pathways, continuing the underlying pathology of the disease. Each of these metabolites derived from oxidized PUFAs are encompassed by the term “oxidized PUFA product(s)”.

### Disease Progression

When a patient is diagnosed with a specific neurodegenerative disease, the clinician monitors the patient's rate of disease progression both before treatment as described herein (the “natural history”) and then again after a given period of time after initiation of treatment. As above, the loss of functionality relates to the accumulation of oxidized PUFA products that arise from the inability of regulatory enzymes to neutralize sufficient amounts of these products which results in accumulation of these oxidized products in the cells followed by cell dysfunctionality and subsequent death. The faster the accumulation these products, the faster the loss of functionality.

The treatment described herein provides for a steady increase in the amount of deuterated arachidonic acid in the patient's cells, including the at-risk neurons, over time until the concentration reaches an amount that reduces the amount of oxidized PUFA products in the neurons. As this steady increase is variable from patient to patient, a preferred embodiment for the methods described herein include monitoring the patient for appropriate uptake of deuterated arachidonic acid into the at-risk neuron particularly when a prodrug of arachidonic acid is administered. Since the neurons are inherently incapable of being monitored during treatment, the clinician is required to assess therapy in proxy cells. However, the data to date suggests that the concentration of deuterated arachidonic acid in such proxy cells does not correlate 1:1 with that in the neurons. As shown in the Examples, a patient treated with deuterated arachidonic acid for a given period of time and a given dosing of deuterated linoleic acid evidenced a higher concentration of deuterated arachidonic acid in the cerebral spinal fluid (CSF) than that found in the red blood cells. Nevertheless, as shown in the Examples, the concentration of deuterated arachidonic acid in red blood cells can be used as a standalone marker as evidence of a therapeutic concentration in the at-risk neurons.

Still further and as also shown in the examples, stabilization of the neurons is achieved when the concentration of deuterated arachidonic acid in the red blood cells reaches at least about 12% based on the total concentration of arachidonic acid, including deuterated arachidonic acid, in the red blood cells, and preferably from about 12% to about 30%. Indeed, at that concentration, clinical studies have demonstrated improvement in vital functions occurs. The extent of disease progression is measured by the reduction in the rate loss of functionality over a set period of time as compared to the natural history of the patient. The greater the reduction in the rate of loss of functionality, the greater the degree of therapy.

As an example of establishing neuron stability in the case of ALS, reference is also made to the Examples, There, a standard test referred to as ALSFRS-R was used to determine the rate of loss of muscle functionality over time, which is then used to measure the rate of disease progression. This test has 12 components, each of which are measured on a 0 (worse) to 4 (best) scale. The ability of a drug to attenuate the rate of disease progression evidences its efficacy and, in the Example, disease progression using a dosing regimen as described herein reduced disease progression by 90%. Even a modest reduction in the rate of functionality loss is considered significant.

Once therapy with 11,11-D2-linoleic acid or ester thereof is initiated, the buildup of 13,13-D2-arachidonic acid in vivo is an incremental process limited by the patient's physiology, the amount of PUFAs consumed as well as the turnover rate of arachidonic acid in the patient. In general, only about 10% of the linoleic acid consumed is converted to arachidonic acid by in vivo processes. On the other hand and as shown in the examples, the concentration of 13,13-D2-arachidonic acid in red blood cells required to achieve therapy in the at-risk neurons ranges from about 12% to about 30%, and in one preferred embodiment, from about 15% to about 20%, based on the total amount of arachidonic acid found in these cells (including the deuterated arachidonic acid). Stated differently and using red blood cells as a proxy for determining therapy, when a patient achieves at least about 12% concentration of 13,13-D2-arachidonic acid in red blood cells, the accumulation of oxidized arachidonic acid in the neurons is stabilized to a level where the impaired regulatory enzymes are capable of neutralizing a large portion to substantially all of the oxidized products. In turn, this stabilizes one or more vital functions in the patient and, in some cases, acts to restore a portion of the functionality previously lost.

Without being limited to any theory, the progression of a neurodegenerative disease correlates to a loss of function in the individual neurons due to accumulation of oxidized PUFAs. As concentration of these oxidized products increases to a non-recoverable level, the diseased neurons will initiate a regulated cell death process. Therapeutic intervention prior to initiation of the regulated cell death process or pathway stabilizes these neurons and limits further loss of functionality and death. As stabilization continues, these neurons are capable of improving upon the function that was previously lost. Regardless of the theoretical aspects, this partial rebound in neuronal function has never been established before and represents a major breakthrough in treating neurodegenerative diseases. Still further, this improvement has been shown to improve life expectancy of the patient, as causes of death associated with disease progression which is associated with lost neural functionality are reduced or arrested.

In order to achieve such concentrations, it is necessary to maintain the patient's dosing levels as described herein. This is because, unlike conventional drug therapy where the drug has a very short half-life in vivo, the mechanism of action of deuterated arachidonic acid relies upon achieving and maintaining a certain concentration of deuterated arachidonic acid in the at-risk neurons. However, arachidonic acid is distributed systemically throughout the body albeit preferentially to the brain. Moreover, the buildup of the concentration of deuterated PUFAs is a slow process, taking months to achieve. This means that the concentration of deuterated arachidonic acid in the at-risk neurons as a percent of total arachidonic acid slowly increases until it reaches a therapeutic level.

Given the rate of loss of functionality in patients with neurodegenerative diseases can be quite rapid, any dosing regimen employed must address the patient's need for an onset of therapy that occurs as promptly as possible to preserve as much neuronal function for the patient for as long as possible. Hence, any therapy for treating such neurodegenerative diseases must be effective as soon as practical, preferably within at least 90 days, more preferably within at least 45 days from start of therapy, and even more preferably within a month or less, thereby retaining as much of the patient's functionality as possible and furthermore providing for substantial reductions in the rate of disease progression.

### Compound Preparation

Deuterated 11,11-D2-linoleic acid is known in the art. See, e.g., pubchem.ncbi.nlm.nih.gov/compound/124037379. Other deuterated linoleic acid or esters thereof are either commercially available or are well known in the art. For example, 11,11-D2-linoleic acid, 8,8,11,11-D4-linoleic acid and other deuterated PUFAs are known in the art. See, e.g., U.S. Pat. Nos. 10,052,299 and 10,730,821 both of which are incorporated herein by reference in their entirety. In addition, 8,8-D2-linoleic acid and 11-D1-linoleic acid, Hill, et al., Free Radic. Biol. Med., 53(3):893-906 (2012) which is also incorporated herein by reference in its entirety. Still further deuterated arachidonic acid esters, a prodrug for deuterated arachidonic acid are known in the art. Exemplary known deuterated arachidonic acids include 7,7-D2-arachidonic acid, 13,13-D2-arachidonic acid, 10,10-D2-arachidonic acid, 7,7,10,10-D4-arachidonic acid, 7,7,13,13-D4-arachidonic acid, 10,10,13,13-D4-arachidonic acid, and 7,7,10,10,13,13-D6 arachidonic acid are disclosed by Shchepinov, et al., Molecules, 28(12):31 et seq. (2018). Still further, other deuterated arachidonic acid compounds are known in the art. Conversion of each of these PUFAs into the corresponding esters are well known in the art.

### Methodology—11,11-D2-Linoleic Acid or Ester Thereof

In one embodiment, the methods described herein comprise the administration of a deuterated linoleic acid or an ester thereof that converts to a deuterated arachidonic acid in vivo to provide for deuteration at least at one or more bis-allylic sites. For example, 8,8,11,11-D4-linoleic acid or an ester thereof is a prodrug for 10,10,13,13-D4-arachidonic acid and has its four hydrogen atoms at the bis-allylic carbon atoms at the 10 and 13 positions replaced with deuterium. Likewise, 11,11-D2-linoleic acid or an ester thereof is a prodrug for 13,13-D2-arachidonic acid and has the two hydrogen atoms at it bis-allylic carbon atom at the 13 position replaced with deuterium. Either or both of these can be administered to a patient suffering from a neurodegenerative disease. In vivo, a portion of each of these deuterated linoleic acids is converted to deuterated arachidonic acid as described above. As to 11,11-D2-linoleic acid, the 13,13-D2-arachidonic acid generated therefrom accumulates slowly in the body. This slow accumulation coupled with physiological considerations that provide for differential conversion rates in different patients requires that the 13,13-D2-arachidonic acid is monitored so ensure that a therapeutic concentration is achieved. Such monitoring includes blood tests to ensure that the patient is accumulating 13,13-D2-arachidonic acid consistent with achieving a therapeutic target of a concentration from about 12% to 30% of total arachidonic acid within the red blood cells. If the blood tests evidence levels of 13,13-D2-arachidonic acid in the red blood cells inconsistent with an expected level, based on the dosing levels and the period of time from start of treatment, the clinician can determine if the dosing should be increased, or if a change from the loading dose to the maintenance dose should be delayed. In order to determine whether the levels of 13,13-D2-arachidonic acid are consistent or not with expected levels, comparison of the test results to a standard can be conducted by the attending clinician.

As to the reduction if the rate of loss of vital functions, reference is made to the specific instance of ALS. There, the reduction in the progression of this disease can be readily calculated by using the known and established rates of functional decline measured by the R-ALS Functional Rating Scale-revised after commencement of drug therapy, as compared to the rate of decline prior to drug therapy (natural history of decline in the patient). As the rate of decline is not perceptible on a day-to-day basis, the functional decline is typically measured periodically (e.g. monthly) and is evaluated over a period of time, such as every 1 to 24 months, such as every 3 months, every 6 months, or annually. The period of time may be any value or subrange within the recited ranges, including endpoints.

As set forth in the examples below, the rate of functional decline is predicated on measuring an individual's, or a cohort's, average for the natural history of disease progression. Next, the individual or cohort average for the functional decline is determined at a period of time, such as at 3, 6 or 12 months after initiation of therapy. The rate of decline based on the average of the natural history of the cohort is set as the denominator. The numerator is set as the delta between the rate of the natural history of disease progression and the rate of functional decline after a set period of treatment per this invention. The resulting fraction is the multiplied by 100 to give a percent change. The following exemplifies this analysis.

Cohort A has an average natural history rate of decline in functionality of 28 annualized for a one (1) year period. Six (6) months after initiation of treatment per this invention, Cohort A an annualized average rate of decline in functionality has dropped to 14. This provides a delta of 14 degrees. So, using 14 as the numerator and 28 as the denominator and then multiplying result by 100, one obtains a reduction in the annualized rate of decline of 50 percent.

Provided are several embodiments for the administration of deuterated linoleic acid or ester thereof (e.g.11,11-D2-linoleic acid ethyl ester) to patients for treating this neurodegenerative disease which is a model for other neurodegenerative diseases treatable with deuterated arachidonic acid generated in vivo from deuterated linoleic acid or an ester thereof. In some embodiments, an adult patient is administered a loading dose of deuterated linoleic acid or an ester thereof for a period of time. In embodiments, the loading dose is between about 6 grams and about 12 grams per day. In embodiments, the loading dose is between about 7 grams and about 12 grams per day. In embodiments, the loading dose is between about 8 grams and about 12 grams per day. In embodiments, the loading dose is between about 9 grams and about 12 grams per day. In embodiments, the loading dose is between about 6 grams and about 11 grams per day. In embodiments, the loading dose is between about 6 grams and about 10 grams per day. In embodiments, the loading dose is between about 6 grams and about 9 grams per day. In embodiments, the loading dose is about 9 grams per day. The dose may be any value or subrange within the recited ranges, including endpoints.

In embodiments where the patient is a juvenile (i.e., under 18 years old and less than about 40 kg in weight), the loading dose may be about 2 to 5 grams of deuterated linoleic acid or an ester thereof (e.g. 11,11-D2-linoleic acid ethyl ester) per day. In some embodiments, the loading dose is between about 2 grams and about 4 grams per day. In some embodiments, the loading dose is between about 3 grams and about 5 grams per day. In on preferred embodiment, the loading dose is about 4 grams per day. The dose may be any value or subrange within the recited ranges, including endpoints.

In some embodiments, the loading dose is administered for between about one month and about 12 months. In some embodiments, the loading dose is administered for between about 1 month and about 10 months. In some embodiments, the loading dose is administered for between about 1 months and about 6 months. In some embodiments, the loading dose is administered for between about 2 months and about 12 months. In some embodiments, the loading dose is administered for between about 4 months and about 12 months. In some embodiments, the loading dose is administered for 12 months or longer. The length of time may be any value or subrange within the recited ranges, including endpoints.

In some embodiments, a patient, e.g. an adult patient, is administered a maintenance dose of deuterated linoleic acid or ester thereof (e.g. 11,11-D2-linoleic acid ethyl ester) for a period of time. In embodiments, the maintenance dose is between about 3 grams and about 8 grams per day. In embodiments, the maintenance dose is between about 3 grams and about 7 grams per day. In embodiments, the maintenance dose is between about 3 grams and about 6 grams per day. In embodiments, the maintenance dose is between about 3 grams and about 5 grams per day. In embodiments, the maintenance dose is between about 4 grams and about 8 grams per day. In embodiments, the maintenance dose is between about 5 grams and about 8 grams per day. In embodiments, the maintenance dose is between about 6 grams and about 8 grams per day. The dose may be any value or subrange within the recited ranges, including endpoints.

In some embodiments, the loading dose is administered between meals. In some embodiments, the dosing is done with food, e.g. during meals.

In some embodiments, whether the patient has a therapeutic level of deuterated arachidonic acid incorporated into the patient's neurons is determined. In some embodiments, the level of deuterated arachidonic acid is determined based on the amount of deuterated arachidonic acid in the patient's red blood cells. In some embodiments, the therapeutic target is at least about 12% deuterated arachidonic acid, based on the total amount of arachidonic acid in the red blood cells (e.g., in a blood sample). In some embodiments, the therapeutic target is at least about 15%. In some embodiments, the therapeutic target is at least about 20%. In some embodiments, the therapeutic target is between about 12% and about 30% deuterated arachidonic acid, based on the total amount of arachidonic acid in the red blood cells (e.g., in a blood sample). In some embodiments, the therapeutic target is between about 12% and about 25% deuterated arachidonic acid, based on the total amount of arachidonic acid in the red blood cells (e.g., in a blood sample). In some embodiments, the therapeutic target is between about 15% and about 30% deuterated arachidonic acid, based on the total amount of arachidonic acid in the red blood cells (e.g., in a blood sample). In some embodiments, the therapeutic target is between about 15% and 20% deuterated arachidonic acid, based on the total amount of arachidonic acid in the red blood cells. The amount may be any value or subrange within the recited ranges, including endpoints.

In embodiments, the deuterated linoleic acid or an ester thereof (e.g. 11,11-D2-linoleic acid ethyl ester) may be administered using any dosing regimen. For example, the deuterated arachidonic acid may be administered daily, every two days, every 3 days, etc. In embodiments, the deuterated arachidonic acid may be administered for one week, 2 weeks, 3 weeks, 4 weeks or more, followed by a period of time where no deuterated arachidonic acid is administered (e.g., 1 week, 2 weeks, 3 weeks or more). In embodiments, the deuterated arachidonic acid may be administered for a period of time until the therapeutic level is reached, and then administered less frequently. For example, the deuterated arachidonic acid may be administered daily until the therapeutic level is reached, and then administered every 2, 3, 4, 5, 6, or 7 days, or longer, to maintain the therapeutic level.

### Combinations

The therapy provided herein can be combined with other treatments used to treat neurodegenerative diseases. In one embodiment, deuterated linoleic acid or an ester thereof (including 11,11-D2-linoleic acid ethyl ester) can be used alone. Alternatively, the deuterated linoleic acid or ester thereof is 8,8,11,11-D4-linoleic acid ethyl ester or a combination, e.g. with 11,11-D2-linoleic acid ethyl ester, can be used. In either case, a fraction of the total amount of these deuterated linoleic acids is converted in vivo, e.g. to either 13,13-D2-arachidonic acid or to 10,10,13,13-D4-arachidonic acid. Both of these can be administered in the loading dose or the maintenance dose as a prodrug for conversion to deuterated arachidonic acid.

The total amount of deuterated arachidonic acid so converted is a fraction of the amount of deuterated linoleic acid or ester thereof administered. This fractional conversion allows the clinician to titrate the amount of deuterated arachidonic acid generated in vivo upward or downward by administering deuterated linoleic acid or ester thereof. This is particularly the case for the maintenance dose where minimal amounts of deuterated arachidonic acid may be required, as the literature recognizes that the amount of biogenerated arachidonic acid is low. See, e.g., Tallima, et al., J. Adv. Res., 11:33-41 (2018).

In another embodiment, a combination therapy can employ a drug that operates via an orthogonal mechanism of action relative to the methods described herein. Suitable drugs for use in combination include, but not limited to, antioxidants such as edaravone, idebenone, mitoquinone, mitoquinol, vitamin C, or vitamin E, provided that none of these anti-oxidants interfere with the therapeutic action of the deuterated linoleic acid or ester thereof.

### Pharmaceutical Compositions

The specific dosing of deuterated linoleic acid or ester thereof is accomplished by any number of accepted modes of administration. As noted above, the actual amount of the drug used in a daily or periodic dose per the methods of this invention, i.e., the active ingredient, is described in detail above. The drug can be administered at least once a day, preferably once or twice or three times a day.

This invention is not limited to any particular composition or pharmaceutical carrier, as such may vary. In general, compounds of this invention will be administered as pharmaceutical compositions by any of a number of known routes of administration. However, orally delivery is preferred typically using tablets, pills, capsules, and the like. The particular form used for oral delivery is not critical but due to the large amount of drug to be administered, a daily or periodic unit dose is preferably divided into subunits having a number of tablets, pills, capsules, and the like. In one particularly preferred embodiment, each subunit of the daily or periodic unit dose contains about 1 gram of the drug. So, a daily or periodic unit dose of 9 grams of the drug is preferably provided as 9 sub-unit doses containing about 1 gram of the drug. Preferably, the unit dose is taken in one, two or three settings but, if patient compliance is enhanced by taking the daily or periodic unit dose over 2 or 3 settings per day, such is also acceptable.

Pharmaceutical dosage forms of a compound as disclosed herein may be manufactured by any of the methods well-known in the art, such as, by conventional mixing, tableting, encapsulating, and the like. The compositions as disclosed herein can include one or more physiologically acceptable inactive ingredients that facilitate processing of active molecules into preparations for pharmaceutical use.

The compositions can comprise the drug in combination with at least one pharmaceutically acceptable excipient. Acceptable excipients are non-toxic, aid administration, and do not adversely affect the therapeutic benefit of the claimed compounds. Such excipient may be any solid, liquid, or semi-solid that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Other suitable pharmaceutical excipients and their formulations are described in Remington's Pharmaceutical Sciences, edited by E. W. Martin (Mack Publishing Company, 18th ed., 1990).

The compositions as disclosed herein may, if desired, be presented in a pack or dispenser device each containing a daily or periodic unit dosage containing the drug in the required number of subunits. Such a pack or device may, for example, comprise metal or plastic foil, such as a blister pack, a vial, or any other type of containment. The pack or dispenser device may be accompanied by instructions for administration including, for example, instructions to take all of the subunits constituting the daily or periodic dose contained therein.

The amount of the drug in a formulation can vary depending on the number of subunits required for the daily or periodic dose of the drug. Typically, the formulation will contain, on a weight percent (wt %) basis, from about 10 to 99 weight percent of the drug based on the total formulation, with the balance being one or more suitable pharmaceutical excipients. Preferably, the compound is present at a level of about 50 to 99 weight percent.

In preferred embodiment, the drug is encapsulated in a capsule without the need for any excipients such as stabilizers, antioxidants, colorants, etc. This minimizes the number of capsules required per day by maximizing the volume of drug in each capsule.

### EXAMPLES

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying figures. Such modifications fall within the scope of the appended claims. In these examples, the following terms are used herein and have the following meanings. If not defined, the abbreviation has its conventional medical meaning.
D2-AA=13,13-D2-Arachidonic Acid
AA=Arachidonic Acid
ALSFRS-R=Revised ALS Functional Rating Scale
BQL=Below Quantitation Limit
CNS=Central Nervous System
CSF=Cerebral Spinal Fluid
D2-LA=11,11-D2-Linoleic Acid (aka “drug”)
LA=Linoleic Acid
LPO=Lipid peroxidation
PK=Pharmacokinetics
RBC=Red Blood Cells
SAE=Serious Adverse Events

### Example 1—Determination of D2-AA Concentrations in RBCs and Spinal Fluid/Neurons in a Single Patient

This example determines the relative concentration of D2-AA in the CSF and in RBCs in order to determine a correlation between these two concentrations. Specifically, a patient was provided with a daily dose of 9 grams of D2-LA ethyl ester (which is 8.64 grams of active discounting for impurities and removal of the ethyl ester) over about a six-month period. Periodic samples of blood and SF were taken and the concentration of both D2-LA and D-2-AA in both the RBCs and the SF were measured. In all cases, the D2-AA was obtained by deacylation of the ethyl ester of linoleic acid in the gastrointestinal tract followed by conversion of a portion of the D2-LA in vivo to D2-AA.

TABLE 1

| | Concentration | Concentration | Ratio of D2-LA |
|---|---|---|---|
| | of D2-LA in | of D2-AA in | to D2-AA in |
| Time | CSF | CSF | CSF |
| 1 month | 19.8% | 8% | 2.5:1 |

The results found in Table 1 show that the concentration of D2-AA in the cerebral spinal fluid is already 8% based on the amount of arachidonic acid+deuterated arachidonic acid. Next, Table 2 shows that the concentration of D2-LA and D2-AA in the RBCs at 3 months and 6 months for the same patient.

TABLE 2

| | Concentration | Concentration | Ratio of D2-LA |
|---|---|---|---|
| | of D2-LA in | of D2-AA in | to D2-AA in |
| Time | RBCs | RBCs | RBCs |
| 3 months | 34.7% | 11.8% | 2.9:1 |
| 6 months | 34.5% | 16.7% | 2.1:1 |

Note that while the concentration of D2-LA in RBCs at 3 months is about double that in CSF at 1 month, the concentration of D2-AA in the RBCs at 3 months is less than 50% greater than D2-AA in the spinal fluid at 1 month. This suggests that a disproportionate amount of D2-AA is being shunted to the CNS than to RBCs. Also note that the concentration of D2-LA in RBC's at 6 months is less than that at 3 months while the amount of D2-AA increases during the same timeframe. This suggest that more D2-LA is being converted to D2-AA over time because a portion of D2-LA previously administered has accumulated in the patient and acts as reservoir for conversion to D2-AA. If so, then reducing the amount of D2-LA when transitioning from a loading dose to a maintenance dose will have minimal impact on the increase in D2-AA in the period after the transition.

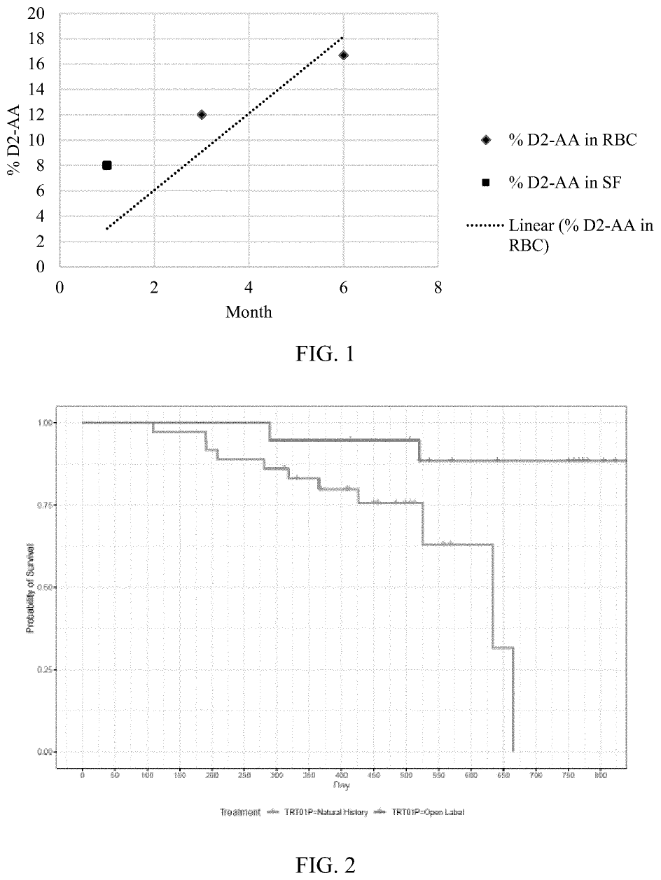

Since the amount of D2-AA is increasing over time in an incremental fashion based on the bioconversion of D2-LA, one can assume a fairly linear rate of increase due to rate limiting steps in the bioconversion. This is shown in FIG. 1, where the solid line is set by the concentrations of D2-AA at 3 months and 6 months and then extrapolated back to start of therapy (0 months). The value for the D2-AA in RBC's at 1 month is estimated from this relationship. The amount shown for 1 month in the CSF is also provided (open circle).

Based on the above, one can see that the data to date suggests that the amount D2-AA at 1 month in RBCs would be about 3 percent as compared to 8% for the amount of D2-AA in the CSF. Accordingly, this data suggests that the concentration the body shunts more of the AA (including D2-AA) into the CSF (and hence the neurons) as compared to RBCs and likely other reporter cells.

### Example 2A—Short-Term Determination of D2-AA Concentrations in a Cohort of 14 Patients

In this example, children suffering from INAD were treated with a daily loading dose of 3.84 grams of D2-LA ethyl ester for 30 days, followed by a daily maintenance dose of 2.88 grams of D2-LA ethyl ester. Given the age and weight of these children, such is assumed to be substantially equivalent to an adult loading dose of about 9 grams per day for an adult patient and a maintenance dose of about 5 grams per day for an adult patient. For the purposes of this application, a loading dose of about 9 grams per day for an adult is construed to be 3.84 grams per day for a child and a maintenance dose of 5 grams per day for an adult is construed to be 2.88 grams per day for a child.

This example also determines the concentration of D2-AA in RBCs. Specifically, a cohort of 14 children was provided with a daily dose of 3.84 grams of D2-LA ethyl ester for 1 month followed by 2.88 grams of D2-LA ethyl ester for the remaining six-month period. Blood samples were taken at 3 months for all but 1 child and at 6 months for all children. The concentration of D2-AA in RBCs was measured. In all cases, the D2-AA was obtained by deacylation of the ethyl ester of linoleic acid in the gastrointestinal tract followed by bioconversion of D2-LA in vivo to D2-AA.

At 3 months, the average concentration of D2-AA in the RBCs was determined to be 12% (6.8% low and 16.8% high). At 6 months, the average concentration of D2-AA in the RBCs was determined to be 16.7% (12.0% low and 26.1% high). This differential in concentrations evidences that bioconversion of D2-LA into D2-AA varies due to physiological differences as well as differences in diet, etc. Such differences dictate that testing for D2-AA in patients should be conducted periodically to ensure that patients are achieving the targeted therapeutic concentration of at least 12% D2-AA in red blood cells (based on the entire amount of AA present). Once tested, the results can be compared to a standard concentration curve.

As can be seen, the graph in FIG. 1 provides an illustrative example of a conventional concentration curve albeit, a more suitable curve would have more data points. Nevertheless, such a concentration curve would allow the clinician to compare the patient's rate of D2-AA generated to that curve. If the patient is not bio-generating sufficient D2-AA, the clinician can change the dosing of D2-LA and/or reduce the daily amount of PUFA consumed by the patient.

### Example 2B—Long Term Determination of D2-AA Concentrations in RBCs

Concentrations of D2-LA, its metabolite 13,13-D2-arachidonic acid (D2-AA), and unlabeled LA and AA were quantitated in plasma and red blood cells (RBCs) of INAD patients being treated with D2-LA ester. Samples were collected on Days 0 (pre-dose), and post start of therapy at days 90, 180, 360, and 540 from 19 patients. It was not possible to collect the full complement of samples from all 19 individuals across the full-time course. Accordingly, the data provided herein represents an average from all available samples at a given collection date.

Descriptive statistics for concentrations in both plasma and RBCs were determined at each time point by days from start of therapy and analyte and mean and standard deviation concentration values were evaluated for trends. Samples that were reported as BQL were set to 0 μg/mL for subsequent analyses. Any individual for whom all the samples collected were BQL was not included in the analysis for that analyte and matrix.

Data were assessed based on absolute concentration values as well as the D2 analogues as a percentage of total polyunsaturated fatty acid (PUFA) concentrations: D2-LA/(D2-LA+LA) and D2-AA/(D2-AA+AA). D2-percentages provided an indication of the extent to which the administered D2-LA and metabolite D2-AA were incorporated into the total LA and AA pools, respectively, which is considered the key indicator of attainment of the therapeutic target. See U.S. Provisional Patent Application Ser. No. 63/177,794 (the '794 application) which is incorporated herein by reference in its entirety.

As expected, all concentrations of both D2-LA and D2-AA were BQL on Day 0, prior to administration of, and thus D2-LA and D2-AA percent of total values on Day 0 were 0%. The D2 percent of total values were moderately to highly variable across the time points, with coefficient of variation values for D2-AA and D2-LA ranging from 91% to 140% and 39% to 60% in plasma, and 37% to 78% and 56% to 83% in RBCs, respectively. This evidences a need to monitor the blood levels of patients being treated with D2-LA ester to establish whether the patient is responding appropriately to the therapy or if a change in dosing is required. See, the '794 application supra.

In general, an important source of variability contributing to the highest CV % appeared to be the presence in the data set of several D2 percentage values of 0% (D2 analogue BQL [0 μg/mL] and unlabeled PUFA measurable) or 100% (D2 analogue measurable and unlabeled PUFA BQL [0 μg/mL (micrograms per millilter)].

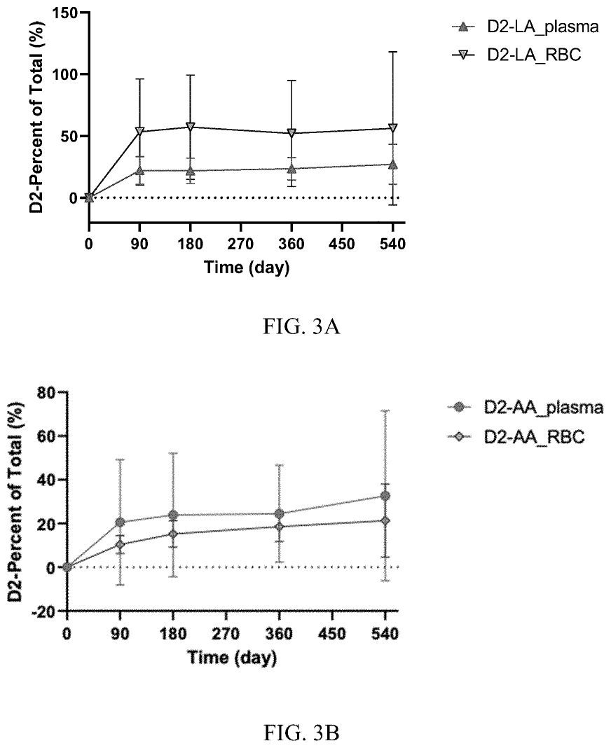

Plots of D2 accretion over the time course are presented for plasma and RBCs for D2-LA in FIG. 3A and D2-AA in FIG. 3B. Specifically, FIG. 3A measured D2-LA concentrations in plasma over 18 months whereas FIG. 3B measured D2-AA concentrations in red blood cells over 18 months. The concentration of D2-LA was based on the total concentration of LA including D2-LA separately in the plasma and then in the red blood cells. Likewise, the concentration of D2-AA was based on the total concentration of AA including D2-AA separately in the plasma and then in the red blood cells.

As can be seen from FIGS. 3A and 3B, measuring D2-LA in RBCs provided a wider range of variability than in plasma. Surprisingly, the reverse was true for D2-AA—measurements were much tighter for D2-AA in RBCs than in plasma. Hence, testing patients for concentrations of D2-AA were determined best to be done with RBCs.

D2-LA as a percentage of total LA increases over the initial time period, appearing to achieve steady state by Day 90 in plasma and Day 180 in RBCs, then remains fairly constant over the ensuing time course through Day 540 in both matrices. From Day 90 to Day 540, mean D2-LA percentages in RBCs ranged from 53% to 56% and were higher than and paralleled those in plasma, which achieved mean D2-LA percentages of 22% to 27%. It is considered that these percentages have sufficiently met the 20% therapeutic target.

D2-LA demonstrates conversion to D2-AA in vivo, as evidenced by the manifestation of increasing D2-AA accretion across time in both plasma and RBCs following repeated administration of D2-LA ester. In contrast to D2-LA, percentage D2-AA values did not reach a stable plateau over the 540 day time course of the study to date, but increase throughout dosing to Day 540. D2-AA percentages are consistently higher in plasma than RBCs, with profiles that are generally parallel. From Day 90 to Day 540, mean D2-AA percentages increase from 21% to 33% in plasma and from 10% to 21% in RBCs. Therefore, the 20% therapeutic target also appears to have been met with D2-AA in plasma and RBCs over the course of the treatment period. This data suggests that the variability of plasma concentrations of D2-AA is less reliable as compared that in RBCs.

The continued accretion of D2-AA throughout the time course may reflect a slow elimination rate. Based on limited data in 5 individual patients who were sampled through a washout period after treatment cessation at 12 months, average elimination half-life estimates for D2-AA were on the order of 70 to 163 days.

### Example 3—Treatment of INAD with 13,13-D2-Arachidonic Acid

A clinical study was completed that tested the efficacy of 13,13-D2-arachidonic (D2-AA) acid in treatment of Infantile Neuroaxonal Dystrophy (INAD) where D2-AA was generated in vivo by bioconversion from 11,11-D2-linoleic acid. This disease is an ultra-rare disorder that is caused by mutations in the PLA2G6 gene that encodes for the PLA2G6 enzyme. This enzyme is responsible for repair of the oxidative damage to PUFAs in lipid bilayers arising from reactive oxygen species by converting damaged PUFAs in the phospholipids into fatty acids. This mutation limits the neutralization capacity of oxidized PUFA products generated in cells such as neurons. As the infant ages, an imbalance is created between the amount of oxidized PUFA products generated and the ability of the damaged PLA2G6 enzyme to neutralize these products resulting in the accumulation of non-neutralize oxidized PUFA products in the neuronal axons. In turn, this leads to loss of neurotransmission in the brain, mitochondrial and neuronal dysfunction, and ultimately in cell death. The disease symptoms usually present between 6 months and 2 years of age and there is often a rapid onset of motor and intellectual regression. Biallelic mutations in the PLA2G6 gene have been identified as the most frequent cause of INAD. However, the same genotype (PLA2G6) causes young onset Parkinson's disease when present in the heterozygous state.

The clinical study consisted of two components as follows:
A first group of 19 patients (1^{st} cohort) were treated during an open label clinical study. Each of these patients were treated with a daily regimen of 3.84 grams of D2-LA ethyl ester followed by a daily maintenance dose of 2.88 grams of D2-LA ethyl ester. Given the age and weight of these children, this dosing is considered to be substantially equivalent to an adult loading dose of about 9 grams per day for an adult patient and a maintenance dose of about 5 grams per day for an adult patient; and
a second or control group of 36 patients (2^{nd} cohort) who were evaluated for the natural history of the disease to whom no drug was administered.

The characteristic regression of acquired motor skills consistent with this disease include delayed/difficulty walking, low muscle tone in the trunk and muscle tightness and weakness in both arms and legs. As the disease progresses, vital functions begin to be compromised, such as swallowing. Swallowing difficulties manifest themselves by aspiration of food into the lungs that leads to pneumonia—an often fatal comorbidity event. Difficulty with neck control are visually apparent as the patient is unable to support lifting the head. In general, mortality occurs by the age of 10.

The clinical study was able to measure changes in life expectancy of treated patients over non-treated patients (control).

The clinical study evaluated disease progression for both loss of vital functionality as well as voluntary muscle control (non-vital functionality). The loss of vital functionality measured survival and pneumonia events. The loss of non-vital functionality measured functional scales to assess the degree of disease progression by functional degradation. In addition, the 1^{st} cohort was also tested for red blood cell concentrations of 13,13-D2-arachidonic acid and any safety issues related to the drug.

In both components, the results from the 1^{st} cohort were first compared against the entirety of the population of the 2^{nd} cohort and then against matched patients from the 2^{nd} cohort (the 19 patients in the treated group were matched to 19 patients in the non-treated group based on similarity of baseline characteristics to the 19 patients in the treatment study).

Mortality rates arising from progression of the disease was monitored in both cohorts. FIG. 2 illustrates a Kaplan Meier Curve illustrating survival curves for the treated patients and all patients in the control. The results evidence a statistically different rate of survival for the treated patients (p=0.014) against control. In turn, this data was extrapolated to provide for an expected increase in life expectancy for the treated patients of about 5 months for each year on therapy.

The following table breaks down these results further by comparing the results for the treated patients against all control patients and then against matched pairs of treated and control patients:

| | Comparison of the | Comparison of the |
|---|---|---|
| | 1^{st} cohort | 1^{st} cohort |
| | against the entire | against the matched |
| | population of the | population of the |
| | 2^{nd} cohort | 2^{nd} cohort |
| Risk of Death | 8.9 × improvement | 5 × improvement |
| Risk of Pneumonia or Death | 5.7 × improvement | 4.1 × improvement |

The results of these comparison evidence a multiple improvement of the treated patients against control in both cases.

The above data evidences vital functionality is significantly improved and stabilized as evidenced by improvements in both risk of death and risk of pneumonia.

In addition, functionality in general results measured spasticity (Modified Ashworth scale) coupled with clinician observations and then parental observations in both the treated patients and control. Across all measurements, the treated patients evidenced a slower progression of the disease by 20-43 percent.

Taken together, these results evidence that treatment of INAD with in vivo generated 13,13-D2-arachidonic acid had a profound impact on the disease progression.

### Example 4—Improvement in Overall Functions in INAD Patients on Expanded Access

Treatment of 2 INAD patients in an expanded access protocol evidenced improvement in both vital function and general function. The treatment protocols described above were used. Each of the patients were evaluated for bulbar function which directly relates to swallowing—a vital function. Patient 1 was evaluated for 12 months from start of therapy and patient 2 for 6 months. The results for each of these patients are as follows:

| | | Patient 1 | | | Patient 2 | |
|---|---|---|---|---|---|---|
| Category | Milestone | Baseline | 6 mos* | 12 mos. | Baseline | 6 mos. |
| Bulbar | Swallows saliva | 0 | 2 | 2 | 1 | 2 |
| | Swallows pureed food | 1 | 1 | 2 | 1 | 1 |
| | Swallows solid food | 1 | 1 | 2 | 1 | 1 |
| | Bite Strength | 1 | 1 | 2 | 1 | |
| | Liquids by feeding tube or syringe | 0 | 0 | 2 | 1 | 1 |

In both patients, the only improvement observed at 6 months relates to swallowing saliva. As is apparent, the inability to swallow saliva results in aspiration into the lungs which leads to a high risk of pneumonia. At 12 months, the first patient evidenced improvement in all of categories of bulbar function suggesting that long term treatment per the methods of this invention cannot only stop further loss of function but actually can result in improvement.

In addition to bulbar function, these two patients were evaluated for fine motor function, gross motor function ocular function and temporo-frontal function. At 6 months, both patients showed improvements by 1 point in holding the head upright (gross motor); tracks human face (ocular) and in each of interacts with parents or examiner, responds to verbal commands, and smiles (temporo-frontal).

Still further, one or the other of the two patients showed a 1-point improvement in:
picks up food with hand or fork (patient 1 at 12 months-fine motor);
stand aided or unaided (patient 1 at 12 months—gross motor function);
walk aided or unaided (patient 2—gross motor);
reaches for objects (patient 2—fine motor);
rings bell (patient 2—fine motor);
places blocks on top of one another (patient 2—fine motor);
nystagmus (patient 1 at 12 months—ocular);
tracks object (patient 1 at 12 months—ocular); and
points to objects in book (patient 2—fine motor).

It is noted that in patient 1, there was a loss of hand grip from start of therapy (+1) to both 6 and 12 months after start of therapy (0). In addition, patient 1 evidenced an increase of +1 (from 0) at 6 months for reaches for objects or bell but lost that improvement at 12 months.

The reversal of a general decline in function is a hallmark of neurodengerative diseases. Eventually, the loss of function includes loss of vital function which ultimately leads to death by co-morbidities such as pneumonia due to loss of the swallowing vital function. As per the above, the methods described herein evidence that certain functionality and especially vital functionality can be restored by long term treatment.

### Example 5—Treatment of ALS with Insufficient Dosing of 11-D2-AA

Patients suffering from ALS were treated with D2-LA over a period of time. The patients were given different dosing amounts of D2-LA and for different dosing periods but did not follow the dosing protocol described in U.S. Ser. No. 17/391,909, which is incorporated herein by reference in its entirety. Some patients were provided 2 grams of 11,11-D-2 LA per day as opposed to the loading dose of 9 grams per day.

Functional scores for each of the patients (ALSFRS-R results) at the end of therapy were compared to the natural history scores at the start of therapy. Based on this comparison, the rate of decline changed from an annualized rate of −14.2+/−4.4 per year pre-treatment to −7.6+/−1.4 during treatment or a 46% reduction (p=0.07, paired t-test for within-subject change in slope). When calculated, the amount of D2-AA in the patients' RBCs averaged at about 3% based on the total amount of AA and D2-AA present. While such evidences that this concentration of D2-AA provided for therapeutic results, it also evidenced that the disease progression was not abated and that there remained oxidized PUFA products that the impaired enzymatic activity was unable to neutralized.

### Example 6—Treatment of ALS with Sufficient Amounts of D2-LA

This example illustrates that the reduction in the rate of disease progression in patients with ALS treated with sufficient doses of 11,11-D2-LA evidences that the amount of oxidized PUFA products has been reduced to a level that allows the otherwise impaired regulatory enzymes are able to neutralize substantially all of these oxidized products now generated. Specifically, a cohort of 3 patients was placed on a dosing regimen consisting of a primer dose of about 9 grams of D2-LA ethyl ester daily for a period of at least 30 days and then transitioning the patients to a maintenance dose of 5 grams of D2-LA ethyl ester.

The functionality of each of the patients was evaluated periodically using the ALSFRS-R protocol. The patients continued on the dosing regimen for a period of 6 months (patient A) or 1 year (patient B) or for 9 months (patient C). Patient C died at the end of 9 months and his death was attributed to factors other than ALS. Before initiation of therapy, the natural history of each patient in the cohort was determined and an average annual rate of functional decline was measured at 21.

The annualized progression of the disease, as measured by an average annual rate of functional decline for all three patients starting at the time that dosing began and terminating at the end of the dosing regimen and then annualized as described above, was measured as 2.1. Using the formula described above, one obtains the following:

(21−2.1)/21×100=90% annualized average reduction in the rate of disease progression.

The specific values for each of the three members of the cohort are as follows in Table 5:

TABLE 5

| | NH Rate of | Functional Rate |
|---|---|---|
| Patient | Decline | Decline During Therapy |
| A | −16 | −3 |
| B | −31 | −2 |
| C | −16 | −1.3 |

NH = Natural History

These results substantiate a very significant rate of reduction in the disease progression using the dosing regimen as per this invention. These results also substantiate that transitioning patients from a primer dose to a maintenance dose maintains the beneficial stabilization in the rate of decline again suggesting that accumulated D2-LA previously administered to the patient acts as depot as the dosing is changed.

As to particulars, these patients treated evidence about a 90% reduction in the rate of disease progression. Compare this to the 46% rate of reduction in the loss of functionality and it is evident that most of the oxidized PUFA product was neutralized in this Example but significantly less was neutralized in Example 5.

The results of the above examples, demonstrate that when the concentration of 13,13-D2-AA reaches about 12% in red blood cells wherein said concentration is based on the total amount of arachidonic acid present, then the patient's rate of loss of functionality decreases to almost zero.

The results for ALS and for INAD demonstrate that the methods set forth herein are applicable to any neurodegenerative disease that is treatable with 11,11-D2-LA and/or with deuterated AA (especially 13,13-D2-AA).

What is claimed is:

1. A method for treating a patient with a neurodegenerative disease which results in neuronal dysfunction and neuronal death, which method comprises administering sufficient amounts of deuterated linoleic acid or ester thereof to said patient for a sufficient period of time such that deuterated arachidonic acid is biogenerated from the deuterated linoleic acid or ester thereof by said patient and is incorporated into the neurons and red blood cells of the patient wherein said sufficient period of time provides for a concentration of deuterated arachidonic acid in red blood cells of said patient of at least about 12% based on the total amount of arachidonic acid present in the red blood cells.

2. A method for stabilizing at least one vital function in a patient with a neurodegenerative disease wherein the at least one vital function has been degraded as a result of said disease, said method comprises:
administering to said patient a sufficient amount of 11,11-D2-linoleic acid or ester thereof such that deuterated arachidonic acid biogenerated from the 11,11-D2-linoleic acid or ester thereof by said patient is incorporated into neurons and red blood cells of the patient; and
continuing said administration of said deuterated linoleic acid or ester thereof until the concentration of deuterated arachidonic acid in red blood cells from the patient is at least about 12 percent based on the total amount of arachidonic acid, including deuterated arachidonic acid, in the red blood cells, whereupon at least one vital function in said patient has been stabilized.

3. The method of claim 1, wherein said concentration of deuterated arachidonic acid in red blood cells from the patient is from about 12 percent to about 30 percent based on the total amount of arachidonic acid, including deuterated arachidonic acid, in the red blood cells.

4. The method of claim 1, wherein the deuterated linoleic acid or ester thereof is administered between meals.

5. The method of claim 1, wherein life expectancy of the patient is increased.

6. The method of claim 2, wherein said concentration of deuterated arachidonic acid in red blood cells from the patient is from about 12 percent to about 30 percent based on the total amount of arachidonic acid, including deuterated arachidonic acid, in the red blood cells.

7. The method of claim 2, wherein at least one vital function in said patient is improved.

8. The method of claim 2, wherein the deuterated linoleic acid or ester thereof is administered between meals.

9. The method of claim 2, wherein life expectancy of the patient is increased.

10. A method for inhibiting cellular dysfunctionality and subsequent cell death due to cellular accumulation of oxidized PUFA products as a result of impaired enzymatic process(es) that limit the neutralization of said oxidized products, said method comprises incorporating deuterated arachidonic acid into said cell and components thereof in sufficient amounts to reduce the amount of oxidized PUFAs generated to a level that said impaired enzymatic processes are capable of neutralizing substantially all of said oxidized products so produced, thereby inhibiting cellular dysfunctionality and subsequent cell death.

11. The method of claim 10, wherein said enzymatic impairment is due to one or more of: genetic defects leading to enzyme with reduced activity; reduction of the amount of enzyme expressed; a reduction in the activity of the enzyme: inability of the cell to produce sufficient enzyme to counter an increasing amount of oxidized PUFA products arising from a diseased condition; or a combination of two or more of these factors.

12. The method of claim 11, wherein the enzymatic impairment is due to age.

13. The method of claim 12, wherein cell death is the result of a regulated cell death pathway.

14. The method of claim 13, wherein the amount of oxidized PUFA products exceeds the ability of said regulatory enzymes to neutralize said oxidized products.

15. The method of claim 13, wherein the regulated cell death pathway is selected from the group consisting of intrinsic apoptosis, extrinsic apoptosis, mitochondrial permeability transition (MPT)-driven necrosis, necroptosis, oxytosis, ferroptosis, and pyroptosis.

16. The method of claim 13, wherein the regulated cell death pathway is mediated by 15-HpETE-PE death signal.

17. A method to treat a neurodegenerative disease in a patient wherein said disease is mediated by neural accumulation of oxidized PUFA products as a result of impaired enzymatic process(es) that limit the amount of said oxidized products that can be neutralized, said method comprises administering a sufficient amount of 11,11-D2-linoleic acid ethyl ester to said patient for a sufficient period of time such that a concentration of 13,13-D2-arachidonic acid in red blood cells ranges from about 12% to about 30% based on the total amount of arachidonic acid, including deuterated arachidonic acid, in the red blood cells, thereby limiting the amount of oxidized PUFA products generated to a level that said impaired enzymatic process(es) are capable of neutralizing substantially all of said oxidized products, thereby treating said disease.

18. The method of claim 1, wherein a concentration of 13,13-D2-arachidonic acid in red blood cells in a blood sample obtained from said patient was assessed.

19. The method of claim 2, wherein a concentration of 13,13-D2-arachidonic acid in red blood cells in a blood sample obtained from said patient was assessed.

20. The method of claim 10, wherein the cell is in a patient and a concentration of 13,13-D2-arachidonic acid in red blood cells in a blood sample obtained from said patient was assessed.

21. The method of claim 18, wherein the concentration of 13,13-D2-arachadonic acid was obtained at a set period after start of therapy and compared to a control.

22. The method of claim 21, wherein the control is a standardized concentration curve.

23. The method of claim 22, further comprising assessing whether the amount of 11,11-D2-linoleic acid or ester thereof administered to the patient should be changed or dose adjusted.

24. The method of claim 23, wherein the amount of 11,11-D2-linoleic acid or ester thereof administered to the patient should be increased if the concentration of 13,13-D2-arachidonic acid in the red blood cells is lower than the control.

25. The method of claim 10, wherein said enzymatic impairment is due to one or more of: genetic defects leading to enzyme with reduced activity; reduction of the amount of enzyme expressed; a reduction in the activity of the enzyme: inability of the cell to produce sufficient enzyme to counter an increasing amount of oxidized PUFA products arising from a diseased condition; or a combination of two or more of these factors.

26. The method of claim 25, wherein the enzymatic impairment is due to age.

27. The method of claim 14, wherein said amount of oxidized PUFA products that exceeds the ability of the regulatory enzyme increases due to disease.
